(19) United States
(12) Patent Application Publication
(10) Pub. No.: US 2011/0245868 A1
(43) Pub. Date: Oct. 6, 2011

(54) Vascular Access Wound Sealing System and Method

(75) Inventors: Rex Teeslink, Augusta, GA (US); Griscom Bettle, Sarasota, FL (US); Talmadge Kelly Keene, Apollo Beach, FL (US)

(73) Assignee: BIOLIFE, L.L.C., Sarasota, FL (US)

(21) Appl. No.: 12/750,168

(22) Filed: Mar. 30, 2010

(51) Int. Cl. A61B 17/04

(52) U.S. Cl. 606/228

(57) ABSTRACT

A wound sealing system and method for closing a vascular access site. The method invisions suturing a single continuous Z-stitch into a skin area around a wound and wound tract while the catheter remains within the vessel; covering the wound and suture holes with a hemostatic powder; tightening and knotting the ends of the suture together in an X configuration, applying finger pressure against the hemostatic powder as the catheter is removed; and twisting the suture ends together to tension the Z-stitch, pulling the skin area into inversion. The wound sealing system includes a powder containment device (PCD) which surrounds wound and catheter and a suture twisting member configured with the PCD to tension the Z-stitch closing the wound and arresting blood flow. The hole in the PCD holds a quantity of the hemostatic agent sufficient to cover the wound and suture holes.

### CROSS-REFERENCE TO RELATED APPLICATIONS

Not applicable

### STATEMENT REGARDING FEDERALLY SPONSORED RESEARCH OR DEVELOPMENT

Not applicable

### INCORPORATION-BY-REFERENCE OF MATERIAL SUBMITTED ON A COMPACT DISC

Not applicable

### BACKGROUND OF THE INVENTION

1. Field of the Invention

This invention relates generally to the field of vascular access procedures and more particularly to a suture tensioning concept for quickly arresting blood flow from a vascular access site.

2. Description of Related Art

Vascular access site closure has a long history of inventive strategies. The standard of care has always been simultaneous direct pressure on the vessel and the insertion site. This typically involves firm pressure over the access site at the skin surface and the vascular site at the surface of the artery or vein. Pressure is held for 10 to 30 minutes based on the blood chemistry and the blood's propensity to clot. During this time the patient typically experiences severe discomfort from the intense pressure.

More recently, pluralities of closure devices have been invented to prevent bleeding at the vascular access site. Some deposit a dose of collagen inside the vascular vessel to prevent bleeding; some leave a mechanical device on the distal side of the access site; others implant a dissolvable “tampon-like” device into the tract between the insertion site and the access site which, upon swelling with blood, physically prevents bleeding from the access site. All of these closure devices can be characterized as having a direct effect on the access site.

A composition for arresting the flow of blood or another protein containing blood fluids flowing from an open wound has been patented by Patterson et al. in U.S. Pat. No. 6,187,347 teaching a substantially anhydrous compound of a salt ferrate which will hydrate in the presence of blood and body fluid to produce Fe⁺⁺⁺ promotes clotting when applied directly over a wound and forming a protective scab attached to the wound to enhance healing thereof. Oxygen is also produced during the reaction.

One aspect of the present invention utilizes the heretofore unrealized virtues of the '347 compound in conjunction with the installation of a catheter and other types of vascular access procedures both at the time of catheter insertion and at the time of removal of the catheter from the vein or artery and skin area of the patient.

More recently, BIOSEAL ADVANCED hemostatic powder by Biolife, L.L.C. of Florida also called (the “powder” hereinafter), a combination of potassium ferrate and acidic cation exchange resin, has been used to form a seal of multi-valent cation-coagulated protein over the access site. This reduced the holding time, for example on arterial sites to 4 minutes, to achieve hemostasis. BIOSEAL ADVANCED is a bone-dry powder that absorbs blood liquid and rejects blood solids. The blood liquids dissolve the potassium ferrate and release a small amount of soluble Fe³⁺. The soluble iron coagulates the accumulating and rejected proteins to create a natural seal over the insertion site, independent of platelet count. The seal is semi-permeable and the powder continues to absorb blood liquids and reject solids. Eventually, the “filter cake” on the proximal side of the seal gets so thick that fluid flow ceases and bleeding stops. When bleeding has ceased, there are no flow events to disrupt the natural clotting at the vascular access site. The blood vessel clots as quickly as the patient's blood chemistry allows. The absorbed cations are replaced with protons from the acidic resin, lowering the pH at the skin/powder interface.

BIOSEAL ADVANCED hemostatic agent significantly reduces the holding time for hemostasis but has no clinically proven effect on the TTA. Importantly, BIOSEAL ADVANCED seals over the insertion site by coagulating blood proteins in situ and then covering the site with a powder at pH 2. The seal acts as a microbial barrier, physically blocking colonizing microbes from infecting the host; the acidic environment adjacent to the seal inhibits microbial growth and kills most pathogens; the patient does not get infected. In a recently published retrospective study of BIOSEAL on PICC lines, the infection rate was reduced 40%.

Once closure has been achieved with any of these devices, the patient has to rest until the physician is confident that he can get up and walk 100 feet. This time is called time to ambulation or TTA. A typical TTA for closure devices is 2-4 hours; for manual pressure, it is 4-6 hours.

The U.S. vascular access industry is huge with more than 6 million arterial accesses per year (2008) splintered across many different service providers. The most common is a catheter lab or an IR lab in a hospital. Recently stand-alone clinics have become a lower cost alternative for routine vascular access procedures. In the U.S., these are regulated by POS11 (Point of Service 11). The gist of this regulation is that no more than 3 patients can be non-ambulatory at any one time. The practical result of POS11 is that procedures are initiated for the first 4 hours of a working day; the latter 4 hours are reserved for TTA and discharge. If the TTA could be reduced to 1 hour, then procedures could be scheduled for 6 hours of a working day, a 50% increase in revenue-generating potential. Thus there is a large economic incentive to reduce TTA.

This driving force has led to the development of internal closure devices. There are, however, several problems with internal closure devices:
1. These devices are safe and effective when the doctor is skilled and careful.
   a. There is a published 10-30% complication rate with these devices.
   b. The devices are used only by the very skilled and shunned by the less skilled.
2. The devices are expensive.
   a. They range in price from $200-$250 per device.
3. Many dialysis patients visit the IR labs 2 to 4 times per year. Devices which are left in the patient take time for biological absorption, typically 90 days. During this interregnum, the vascular access site is not available. If the dialysis patient has to re-visit the IR lab in this time frame, the care the patient needs can be potentially compromised.
4. Many practicing physicians are ethically resistant to devices left inside the body for extended periods of time. This is not a universal concern as ˜33% of all arterial access procedures are closed with a closure device.

There is a real need for a low-cost protocol to shorten TTA to 1 hour and not leave anything in the patient's body while simultaneously preventing external-sourced infections, hematomas and other complications.

Sutures and staples are well known closure means to proximate two sides of a wound. Normal procedure is to make the stitch and pull it tight and then stitch again until the wound is closed. Many inventors have improved on the basic stitch by adding various tensioning means, such as Alghamdi, 20090281569 hereafter '569, Weiss, U.S. Pat. No. 6,471,715 and Cosmetto et al U.S. Pat. No. 5,127,412. '569 teaches a loop through the skin with a slipknot that is then pulled tight to provide tension. '569 teaches this is particularly critical because excess tension can cause cosmetically unacceptable scaring. The key teaching of '569 is direct tension on a suture to close a wound. Weiss and Cosmetto teach mechanical means to provide tension on a suture. One of them teaches a rotary device that converts circular motion into a linear force in-line with the suture.

Shad et al, 2009/0082790, teaches a twist means to facilitate post-operative sternum closure. The suture is placed in a holding device and twisted to bring the internal sides together.

Davis, U.S. Pat. No. 4,773,421, teaches a controlled linear tensioning device because excess tension of the suture can damage underlying skin. This is particularly important for sutures that remain in place for days. Davis points out that his device allows for tension adjustment as the healing process proceeds because frequent cleaning and inspection of the wound is required. Davis also teaches that the angle of the suture to the skin as the suture emerges from tissue is as close to a right angle to avoid the suture cutting into the skin.

Taheri, U.S. Pat. No. 5,919,207, teaches a method of closing an artery by twisting wires in the artery to close the hole, then stapling the site together. The twisted wire is then removed and the artery heals. As an aside, Taheri teaches that the skin opening is approximated with a standard staple. With Taheri, there are two staples on the artery itself and at least one staple on the skin.

Tiefenbrun, U.S. Pat. No. 6,331,182, teaches twisted sutures to close internal tissue. Lafontaine et al, U.S. Pat. No. 5,964,782, teaches that the time to complete the suturing function can result in significant blood loss, particularly on arterial interventions. He goes on to teach about other closure devices that may support clot formation and thrombosis. He goes on to teach that twisting the suture is an effective countermeasure. Like other inventors of closure devices, Lafontaine is dismissive of ways to achieve hemostasis at the access site.

Gao et al, U.S. Pat. No. 6,752,810, teaches that adding tension to a suture is a successful way to approximate two sides of a wound. His device uses an external twister to generate linear tension.

Sancoff et al, U.S. Pat. No. 7,081,124, teaches a suture at an arterial insertion site and then approximating the sides using a twist.

Torque is different than tension. Tension is a linear force aligned with the suture. Torque (or twist) is the force at an angle to tension. Tension and torque together represent the in-line vector and the right-angle vector respectively with respect to the centerline of the suture.

When torque is combined with sutured skin, the skin twists like an Archimedes Screw. Put a finger on the back of your other hand and then rotate 30° to see the Archimedes Screw effect on your skin. When the sutures and sutured skin are rotated clockwise (or counterclockwise) (looking down at the wound site), the lower skin is pulled up towards the torque-inducer. When a vein or artery (artery is used herein to mean either vein or artery) is embedded in the skin adjacent to the “screwed” skin, the arteriotomy is approximated. This is illustrated in Lafontaine '782, FIG. 6c, in which a Y-shaped insertion pattern is twisted into a closed half-spiral. Thus twisting soft tissue to close it is well known.

Doctors work hard to prevent over-tightening sutures because it increases scarring and the potential for skin damage, so that excess tension and especially excess tension and torque is not used in clinical practice. In addition, over-tightening elongates the hole and tends to prevent approximation at the distal end of the insertion site. An enlarged hole is a vector for infection, oozing and complications.

Current best practices for extra-luminal closing of an arteriotomy involve manual pressure. With manual pressure, the nurse or doctor presses a finger on the artery upstream of the arteriotomy while simultaneously pressing on the insertion site. The best practitioners use semi-occlusive pressure such that the radius of the artery at the arteriotomy is little changed. Less skilled practitioners will use heavy pressure at the insertion site that transmits to the arteriotomy and changes the radius of the arteriotomy. Heavy pressure has a large complication rate compared to semi-occlusive pressure including intense pain for the patient.

The prior art teaches:
1. External pressure to close the insertion site, the tract and the vascular access site, or
2. An internal leave-behind unnatural device plus a suture, staple or manual pressure to stop bleeding at the access site, or
3. Manual pressure in combination with a haemostatic powder.

An ideal solution would deliver the single step simplicity of external manual pressure, the short time to hemostasis and TTA of an internal closure device and the low complication rate of using a haemostatic powder plus manual pressure.

The foregoing examples of the related art and limitations related therewith are intended to be illustrative and not exclusive. Other limitations of the related art will become apparent to those skilled in the art upon a reading of the specification and a study of the drawings.

### BRIEF SUMMARY OF THE INVENTION

This invention is directed to a wound sealing system and method for closing a vascular access site. The method invisions suturing a single continuous Z-stitch into a skin area around a wound and wound tract such that the submerged suture is perpendicular to the wound tract while the catheter remains within the vessel; covering the wound and suture holes with a hemostatic powder; tightening and knotting the ends of the suture together in an X configuration, applying finger pressure against the hemostatic powder as the catheter is removed; and twisting the suture ends together to tension the Z-stitch, pulling the skin area into inversion. The wound sealing system includes a powder containment device (PCD) which surrounds wound and catheter and a suture twisting member configured with the PCD to tension the Z-stitch closing the wound and arresting blood flow. The hole in the PCD holds a quantity of the hemostatic agent sufficient to cover the wound and suture holes.

It is an object of the invention to eliminate the need for manual pressure, reduce TTA, reduce scarring and reduce complications. It is also an object of the invention to increase patient satisfaction. It is a further object that the closure means is an extra-luminal strategy with nothing left in the patient at discharge. It is a further object to lower cost.

It is a further object to secure bodily tissue with a Z-stitch encompassing the area defined by the catheter insertion site, and the vascular access site distal to the blood vessel. The ends of the sutures are tied together to form an X. Twisting the external ends to create torque and excessive tension on the stitch further enhances the X. The tissue perpendicular to the subcutaneous stitch indents downward towards the tract and arterial access site and provides low-level continuous pressure on the wound.

It is still a further object to seal the elongated suture holes to prevent oozing, bleeding and microbial colonization.

The following embodiments and aspects thereof are described and illustrated in conjunction with systems, tools and methods which are meant to be exemplary and illustrative and not limiting in scope. In various embodiments one or more of the above-described problems have been reduced or eliminated while other embodiments are directed to other improvements. In addition to the exemplary aspects and embodiments described above, further aspects and embodiments will become apparent by reference to the drawings and by study of the following descriptions.

### BRIEF DESCRIPTION OF THE SEVERAL VIEWS OF THE DRAWING(S)

FIG. 1 is a perspective view of the arterial access after the procedure is complete and closure has begun.

FIGS. 1A to 1D are top plan views of Z-stitch variations forming an inversion aligned with, and perpendicular to, the wound tract.

FIG. 2 is a perspective view of FIG. 1 showing the addition of a nylon powder containment ring (PCD).

FIG. 3 is a perspective view of FIG. 2 showing the addition of a hemostatic powder to the inside of the ring.

FIG. 4 is a perspective view of FIG. 3 showing the start of manual pressure as the sheath is removed.

FIG. 5 is a perspective view of FIG. 4 showing after the sheath is removed and the loose suture ends are tied off.

FIG. 6 is a perspective view of FIG. 5 after knotting the loose ends of the sutures around a twist bar and tied a second time.

FIG. 6A is a top plan view of FIG. 6.

FIG. 6B is a section view in the direction of arrows 6B-6B in FIG. 6A.

FIG. 7A is a section view in the direction of arrows 7A-7A in FIG. 6.

FIG. 7B is a view of FIG. 7A showing the effect of twisting to pull up the 4 suture corners into inversion of the enclosed tissue.

FIG. 7C is a section view in the direction of arrows 7C-7C in FIG. 6 after the suture tightening step in FIG. 7B.

FIG. 7D is a simplified top view of the suture insertion sites and the elongated suture holes after excess suture tension is applied.

FIG. 8 is an exploded perspective view showing the removal of the parts and the removal of the suture just prior to ambulation.

FIG. 9 is a perspective view of FIG. 8 showing the addition of extra powder to seal the now-empty suture sites.

FIG. 10 is a view of FIG. 9 showing holding manual pressure for 15 seconds to seal the 5 puncture sites.

FIG. 11 is a view of FIG. 10 after the closing procedure is complete.

FIGS. 12A to 12C show alternate embodiments of the powder containment device (PCD).

FIGS. 13A and 13B are perspective broken views of alternate twist bars with jam slits to eliminate the need for the second knot.

FIG. 14 is an exploded perspective view of a self-locking suture tensioner and PCD in combination.

FIG. 15 is an exploded perspective view of another embodiment of a self-locking suture tensioner and PCD in combination.

FIG. 16 is an exploded perspective view of the preferred embodiment of the self-locking suture tensioner and PCD in combination.

FIG. 17 is another exploded perspective broken view of FIG. 16.

FIGS. 18 and 19 are perspective views of yet another embodiment of the self-locking suture tensioner and PCD in combination.

Exemplary embodiments are illustrated in reference figures of the drawings. It is intended that the embodiments and figures disclosed herein are to be considered to be illustrative rather than limiting.

### DETAILED DESCRIPTION OF THE INVENTION

A protocol has been developed for implementing the method of the present invention utilizing an anhydrous ferrate and cationic exchange resin composition (the powder) taught in U.S. Pat. No. 6,187,347, the entire teaching of which is incorporated herein by reference.

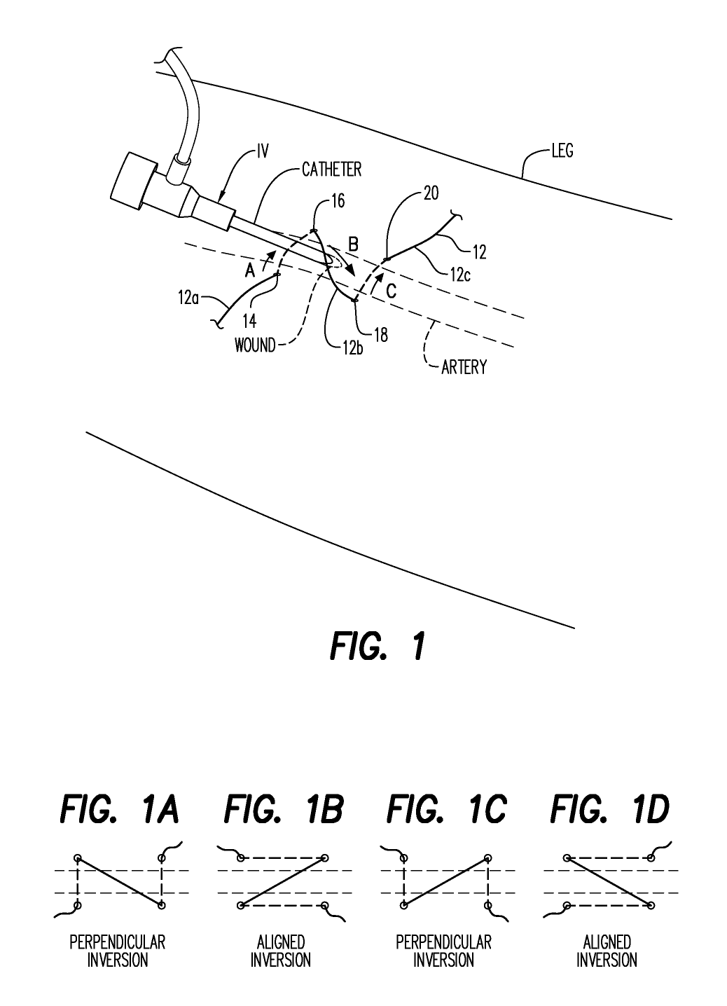

Referring now to the drawings, and firstly to FIG. 1 after an arteriogram or other vascular intervention within an artery of a leg is complete, while the catheter of the I.V. still remains in the artery and the wound tract, a suture 12 is placed into the skin at 14 adjacent the wound and under the skin and emerges at 16. The suture 12 is then diagonally over the catheter and into the skin a second time at 18 to reemerge at a fourth corner 20. Looking down and into the skin, the stitch pattern forms a “Z”, a typical stitch used to suture-close the stump after an appendectomy.

Referring now to FIGS. 1A, B, C, and D, there are four possible ways to configure the Z-stitch. FIGS. 1B and 1D create a skin inversion aligned with the access tract to create semi-occlusive pressure along the axis defined by the access site, the tract and the arteriotomy. FIGS. 1B and 1D are not preferred as the alignment may be off center.

FIGS. 1A and 1C create a skin inversion perpendicular to the tract defined by the access site, the tract and the arteriotomy and this pressure is not applied along the entire length of the wound. FIGS. 1A and 1C are preferred because they assure that pressure is applied across the entire vessel.

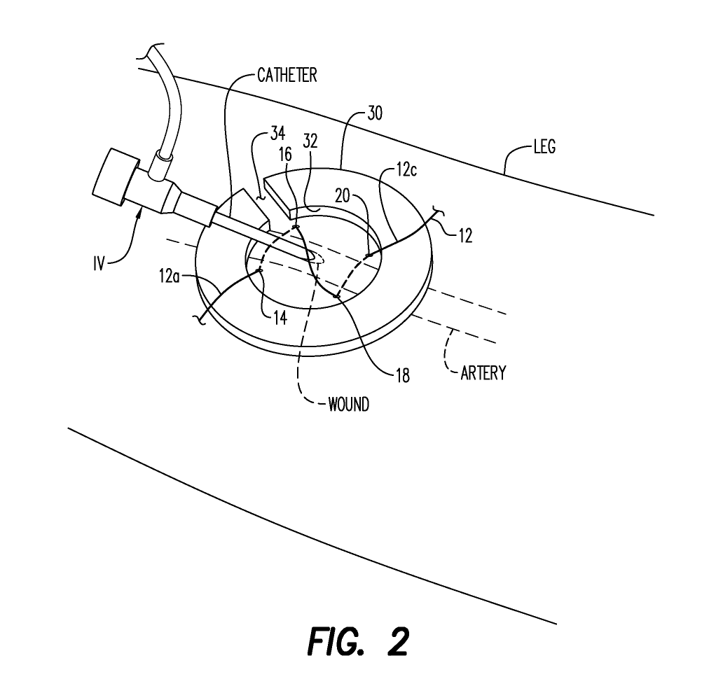

In FIG. 2, a substantially incompressible powder containment device (PCD) 30 is placed around the catheter and the Z stitch 12b. The PCD 30 is formed preferably of substantially incompressible resilient plastic material having a thickness of approximately 0.08″ to 0.40″ having an overall diameter of approximately 2″ with a central hole 32 having a diameter of 1.25″. The diagonal slot 34 is formed through this flat ring-shaped PCD 30 as a clearance to pass over the catheter into the position shown.

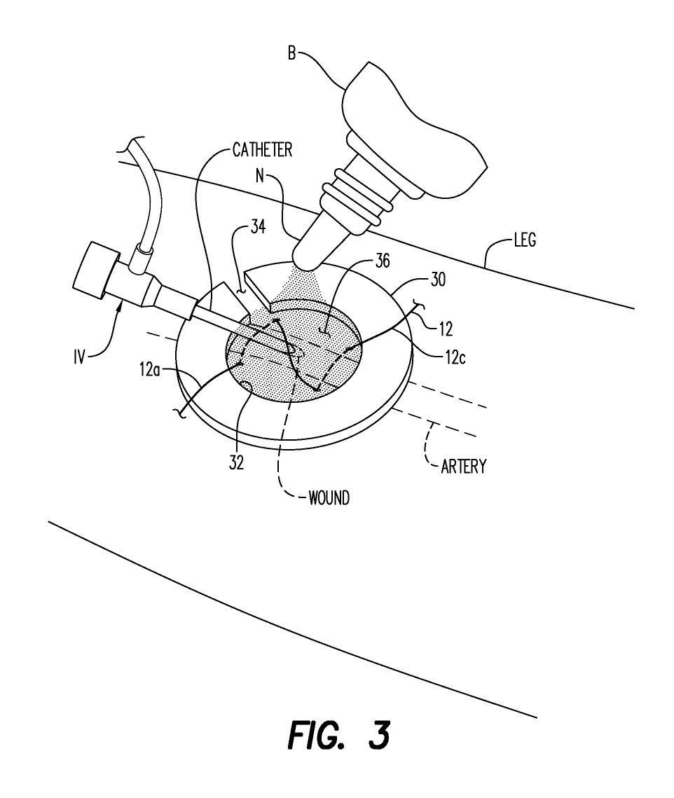

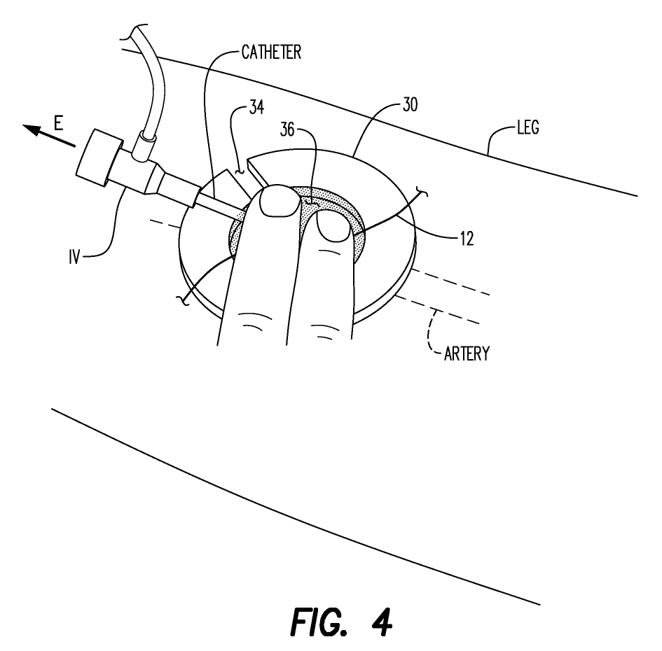

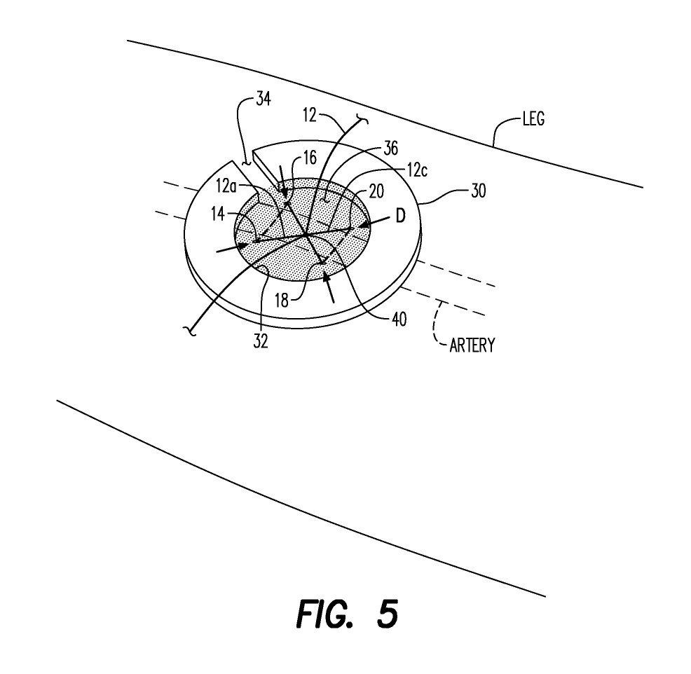

In FIG. 3, a quantity of potassium ferrate/strong acid cationic exchange resin mixture as an anhydrous ferrate compound 36, is poured into the PCD to fill it. In FIG. 4, finger pressure is applied over the powder 36 with gentle pressure while the I.V. with catheter is withdrawn in the direction of arrow C. The ferrate compound is a hemostatic agent which includes an effective amount of a salt ferrate combined with an effective amount of an insoluble cation exchange material, the salt ferrate combining with blood or blood serum to form a trivalent Fe⁺⁺⁺ ion which promotes blood clotting and produces oxygen to reduce the bacteria level at the wound site W. The cation exchange material also forms a protective cover over the wound site as the trivalent Fe⁺⁺⁺ ion is formed. In FIG. 5, both ends 12a and 12c of the suture 12 are then pulled tight in the direction of arrows D at the suture entry points 14, 16, 18 and 20. The suture ends 12a and 12c are then knotted at 40 over the site. Hemostasis is immediate.

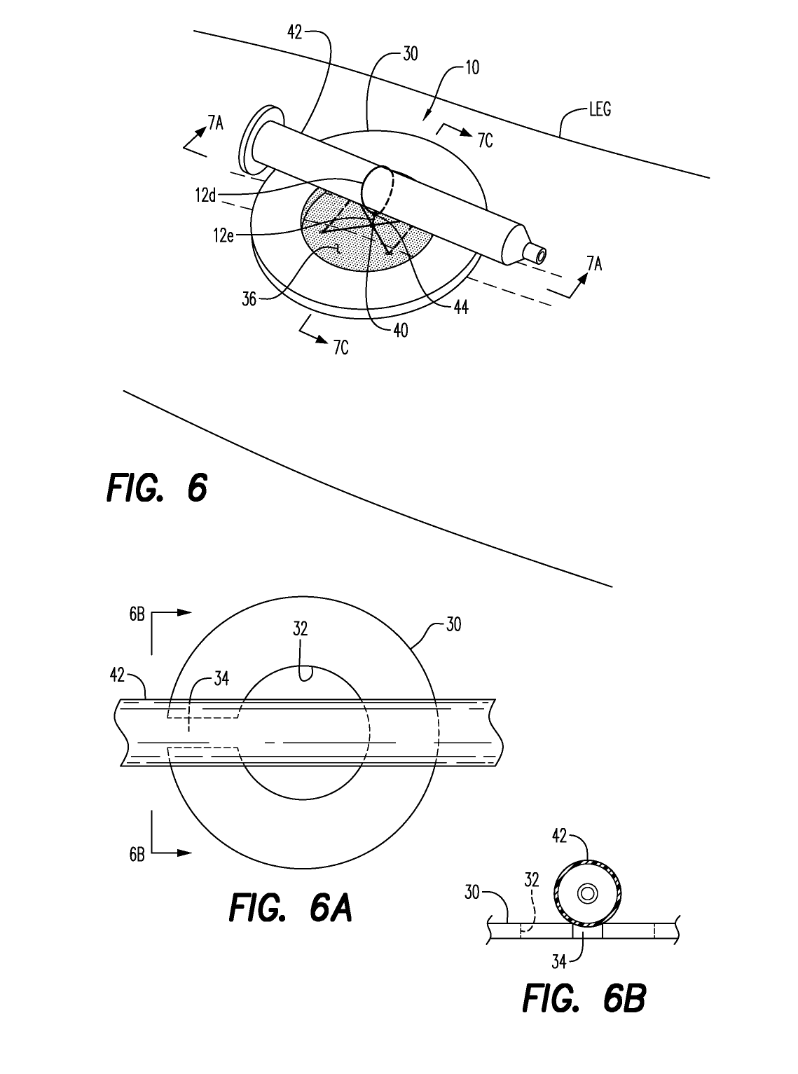

In FIG. 6, a saline syringe is disassembled and the elongated barrel 42 recovered and used as a twist bar or windlass. The loose ends 12a and 12c of the suture 12 distal to the knot 40 over the insertion site are wrapped around the barrel 42 and a second knot is tied at 44, thus forming a “Spanish Windlass”. The barrel 42 is turned clockwise creating a braid 12e of the two ends of the suture 12. As seen in FIGS. 6A and 6B, the twisted suture forms a braid 12e which shortens between knots 40 and 44 thus drawing the barrel 42 against the exposed surface of the PCD 30 and pressing the PCD 30 against the skin. When this shortening-by-twisting effect reduces the distance sufficiently between knots 40 and 44, the barrel 42 becomes parked or wedged into the slot 34 which acts as a detent to keep the barrel 42 and the braid 12e from unwinding.

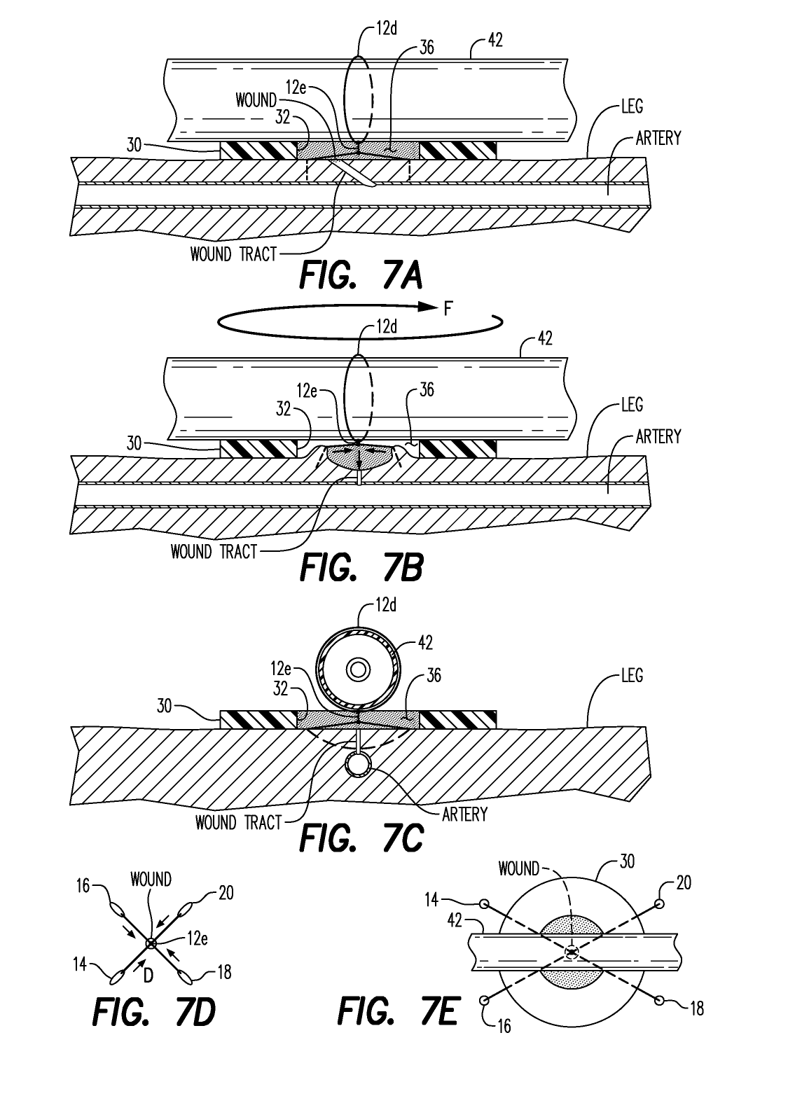

In FIGS. 7A and 7C, as the barrel 42 is turned, the twisted braid 12e winds to shorten the linear distance between the barrel 42 and the wound, creating almost-right-angle tension on the embedded portions of the suture 12. As best seen in FIG. 7B, the right-angle-tension pulls the corners inward and pushes the center down (inversion) towards the artery and along the tract and insertion site. The twisted suture also pulls the barrel 42 and the PCD 30 down toward the skin, creating a physical seal between the PCD 30 and the skin which keeps loose powder 36 from falling out.

In another embodiment, there is an encircling plastic “washer” through which the free ends of the suture are threaded and then the washer is placed over the insertion site. The washer has a detent to bind the windlass means in place after torque has been applied. The barrel can be of any rotatable shape and any material with sufficient strength to withstand the torque.

In a preferred embodiment, the FIG. 4 step is eliminated by pulling the catheter after the FIG. 7 twisting process. This embodiment is preferred because it eliminates a potential weakness, i.e., the time gap between pulling the catheter and achieving inversion. This time gap makes it vulnerable to a hemostasis. Moving FIG. 4 step after inversion eliminates the potential for hemostasis.

### Wound Eversion

According to MacKay-Wiggan et al in Suturing Techniques, May 1, 2009 (Web MD): “The choice of suture technique depends on the type and anatomic location of the wound, the thickness of the skin, the degree of tension, and the desired cosmetic result. The proper placement of sutures enhances the precise approximation of the wound edges which helps minimize and redistribute skin tension. Wound eversion is essential to maximize the likelihood of good epidermal approximation. Eversion is desirable to minimize the risk of scar depression secondary to tissue contraction during healing. Usually, inversion is not desirable and probably does not decrease the risk of hypertrophic scarring in an individual with a propensity for hypertrophic scars.”

The inversion-induced force is on the insertion site, the wound tract and the wound or insertion site. The induced pressure is gentle but consistent and hemostasis is achieved along the entire wound. The barrel 42 is left in place one hour to allow clotting to go to completion. Prior art teaches that right angle tension is undesirable, and it is for extended time, but not for one hour. However, the induced force over the entire site is detrimental to the suture entry sites as they are elongated as seen in FIG. 7D and the open surface area is increased. The hemostatic powder 36 mitigates this issue by sealing the enlarged raw surface area and protects against bleeding and infection. What is key is that the suture sites are elongated for only an hour, much less than the time for complete healing. There is no scarring.

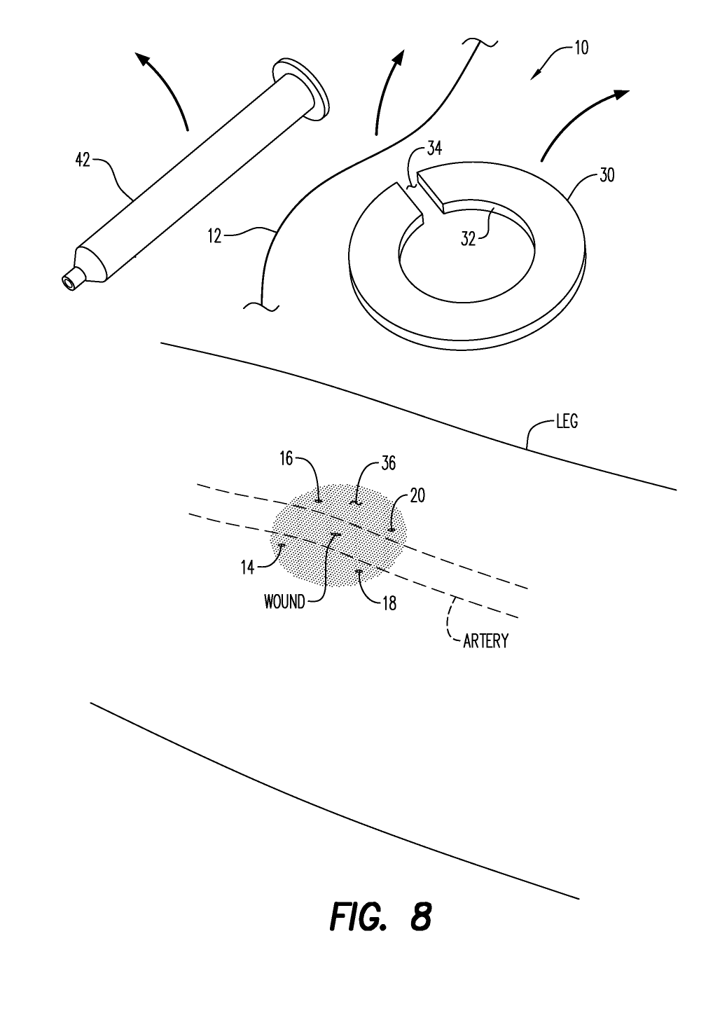

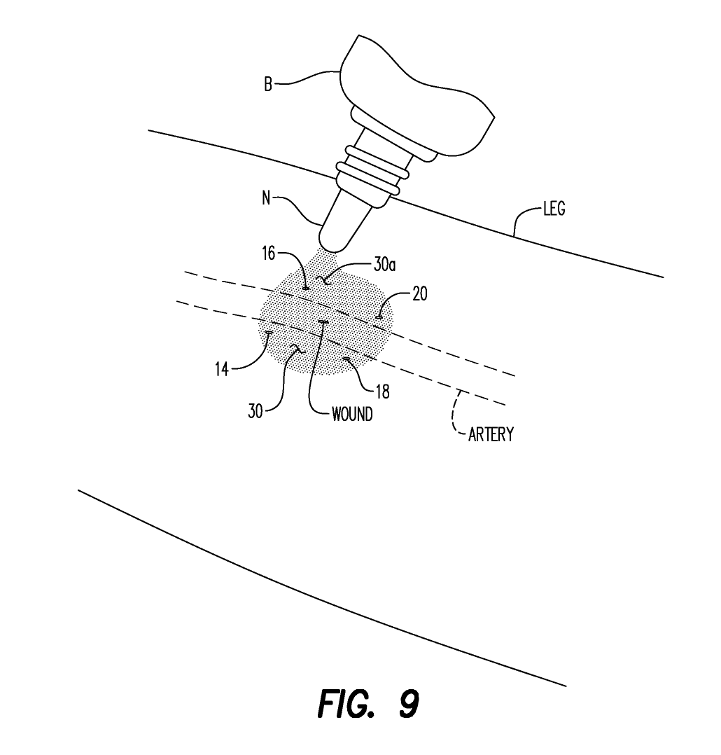

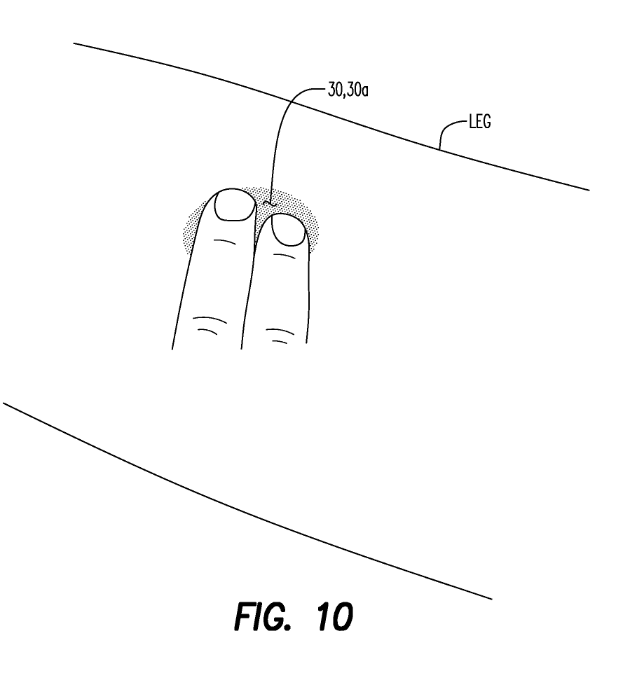

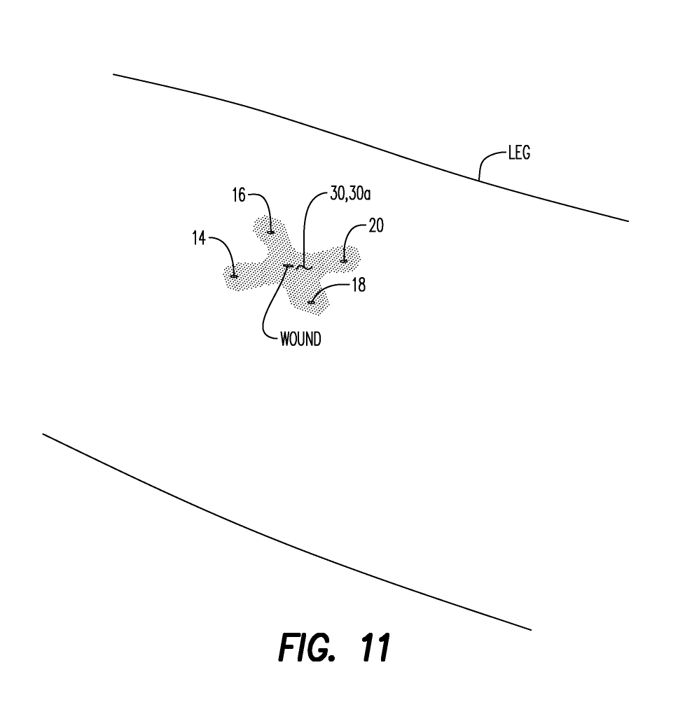

After the hour, the stitches are removed as shown in FIG. 8 as follows:
1. The suture 12 is cut from around the barrel 42;
2. The barrel 42 is removed and the PCD is then removed;
3. The suture 12 is cut one to four times below the knot 40;
4. The one to four suture fragments are pulled out of the skin;
5. Residual hemostatic powder is poured on the suture holes 14, 16, 18 and 20 as shown in FIG. 9 and finger pressure is held to form a seal as seen in FIG. 10.
6. The wound is not bandaged as seen in FIG. 11 because the seal is sufficient.

### Alternate PCD Embodiments

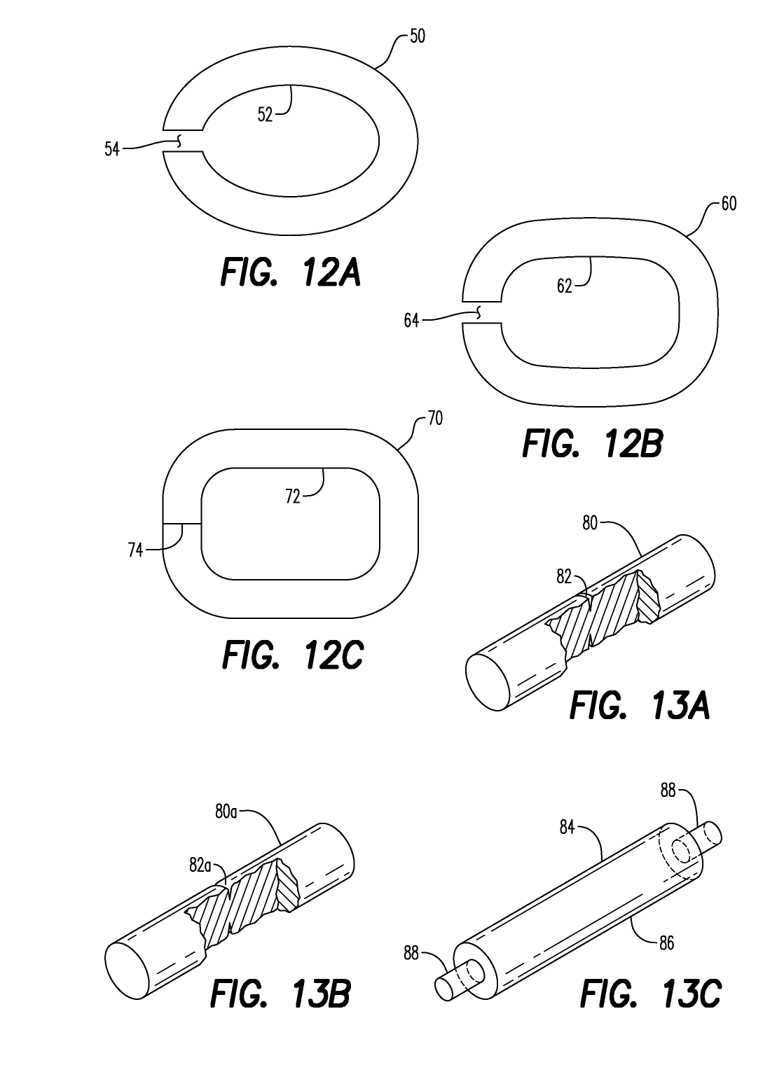

Other embodiments of the PCD facilitate this same methodology. In FIGS. 12a, 12b & 12c, different configurations of the PCD are illustrated at 50, 60 and 70. The PCD 50 includes an oval-shaped hole 52 along with a slot 54 serving as a detent for the twisted-tight barrel 42 as previously described. The PCD 60 includes a racetrack-shaped central hole 62 and the detent slot 64, which, again, facilitates fitting of this PCD around the catheter while still inserted into the vascular leg area. The PCD 70 also includes a central racetrack-shaped hole 72 and a slit 74 which may be resiliently opened for access around the catheter.

In FIGS. 13A and B, dedicated twist bars 80 and 80a each have a jam-fit groove 82 or 82a cut into the cylindrical shape. The physician takes the ends 12 & 12a and jams them into the groove 82 or 82a and then starts twisting. The jam-fit eliminates the need for a second knot.

In FIG. 13C, the twist bar 84 is a “rolling pin-like structure” in which the major diameter 86 is greater than the detent slot 54 or 64 and the minor diameter 88 is less than the detent slot 54 or 64. This way, after inversion is achieved, the minor diameter 88 nests completely in the detent slot 54 or 64 and cannot “unwind” during the TTA.

### Combination PCD and Suture Twister

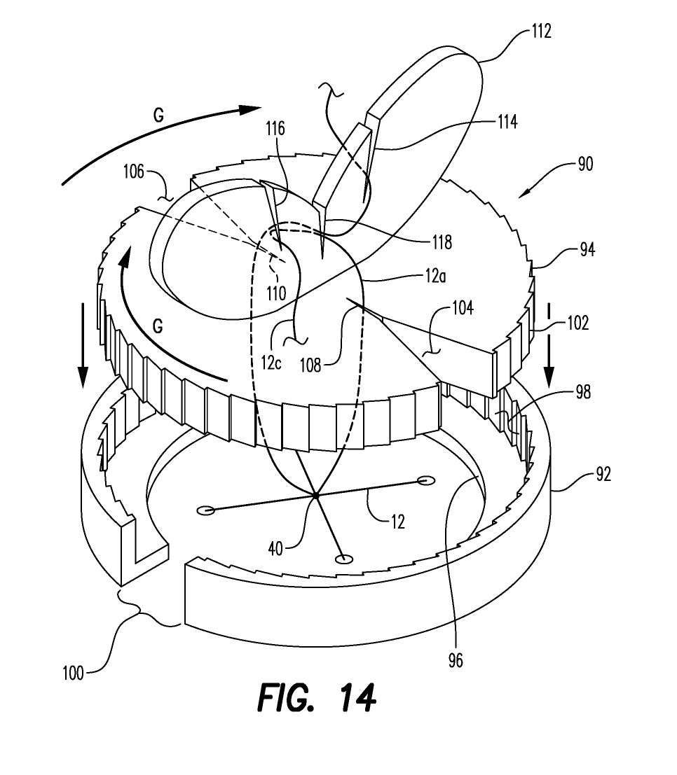

Referring now to FIG. 14, a vascular access wound sealing system as a combination powder containment device and suture twister is there shown generally at numeral 90 and includes a cup-shaped powder containment device 92 and a suture tensioner 94. The PCD 92 includes a central hole 96 and an access slot 100 which function as previously described. The PCD 92 also includes an upright outer wall having inwardly facing internal teeth 98 extending entirely therearound. These teeth 98 mesh with outwardly facing teeth 102 formed around the perimeter of the suture tensioner 94. When the suture tensioner 94 is inserted into teeth meshing engagement with the PCD 92, the suture tensioner 94 is rotatable in only one direction, that being clockwise in the direction of the arrow F.

The suture tensioner 94 also includes an upright finger turning blade 112 connected to a top surface therefore which facilitates the tensioning of the suture 12. The suture ends 12a and 12c are fed from knot 40 into locking notches 104 and 106, respectively, as the suture twister 94 is lowered into tooth engagement with the PCD 92. Thereafter, one of the suture ends 12a is fed through a central locking notch 118 and then through one of the side locking notches 114 or 116 formed through blade 112, while the other suture end 12c is fed firstly through the central locking notch 118 and then through the alternate side-locking notch 116 or 114. Once the suture ends 12a and 12c are lockingly engaged as described and shown, rotation by manual grasping of the finger blade 112 is effected in the direction of arrow G to properly tension the suture 12 as previously described.

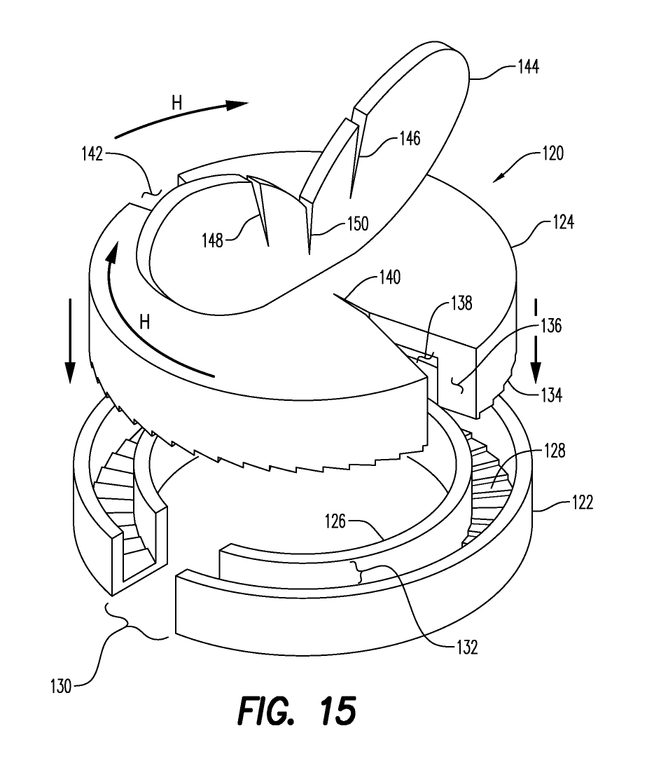

Referring to FIG. 15, the preferred embodiment of the PCD 122 and suture tensioner 124 combination of this wound sealing system is shown generally at numeral 120. The PCD 122 is formed having a U-shaped upwardly opening channel 132 formed along the perimeter thereof having upwardly facing teeth 128 formed at the bottom of this channel 132. The inner wall 126 defines a hole or opening for access to the wound and sutures as previously described, along with notch 130 which provides access around the in-place catheter.

The suture tensioner 124 has downwardly facing teeth 134 which matably engage into teeth 128 of the PCD 122 when the suture tensioner 124 is downwardly positioned into the U-shaped channel 132. The suture tensioner 124 further includes V-shaped notches 138 and 142 having locking ends 140 which secure the suture ends as previously described in FIG. 14. An upright finger blade 144 connected to, and upwardly extending from the upper surface of the suture twister 124, include central and side upright V-shaped locking notches 150, 146 and 148, respectively, for locking interengagement with the suture ends as described with respect to FIG. 14. Rotation in the direction of arrow H tensions the suture 12.

A surface adhering device may also be used to pull the skin from around the access site together while pushing down in the center to create an inversion in the skin and tissue therebeneath. This inversion puts pressure over the hole in the vessel to stop the bleeding from that vessel. This device can be used with or without the hemostatic powder. The device an attach by means of a surface adhesive, mechanical hooks, or the like. The hooks can be designed to barely penetrate the surface of the skin or to penetrate into the tissue beneath. Depth of penetration may be used to control the degree of inversion of the tissue beneath. The device can also be turned, twisting the skin and tissue beneath creating a tortuous track to reduce oozing from said tract.

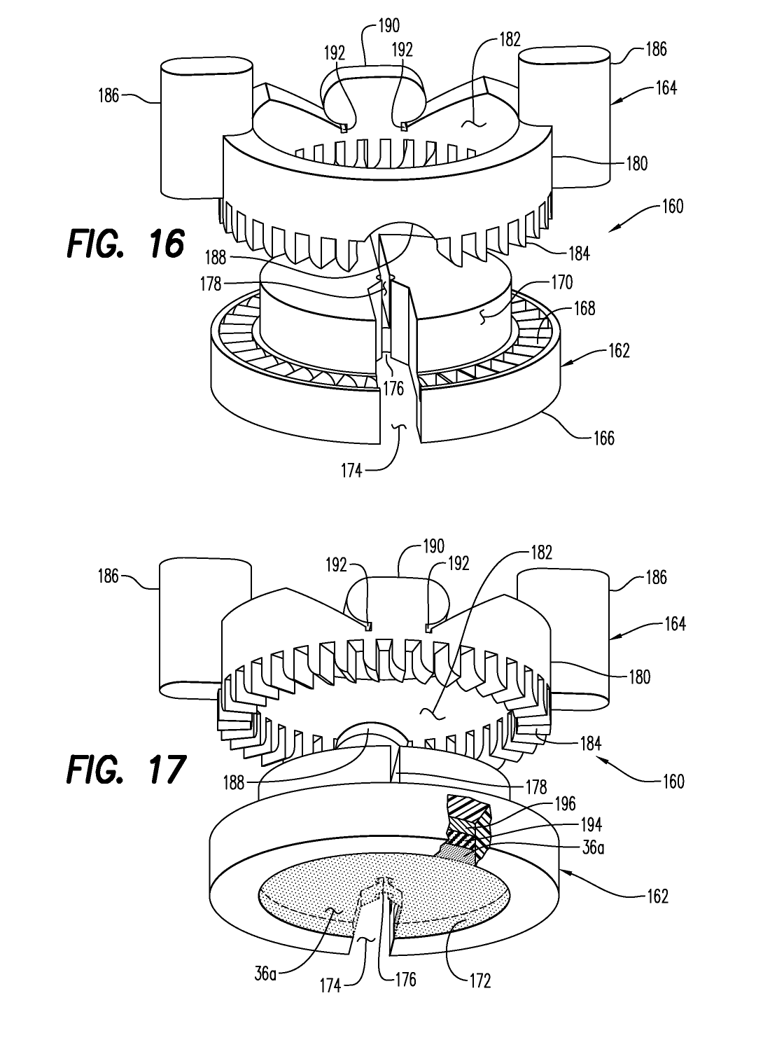

In the FIGS. 15 and 16, a preferred embodiment of the wound sealing system 160 is used to exert low-level continuous pressure similar to the device showed in FIGS. 14 and 15. The FIG. 14 device was found to have a critical defect in that the suture ends slipped in the device 120 and did not retain the required torque. It was also determined that the elapsed time from pulling the sheath (sheath could be any tubular device inserted into the skin) to tying the first knot predicted the frequency of hematomas. A further objective was to eliminate any time lag between application of tension and pulling the sheath.

The FIG. 16/17 wound sealing system 160 works this way:
1. BioSeal Advanced hemostatic powder is improved with the addition of a powderous substance that is attracted to a magnet such as Magnetite, making the entire powder 36a magnetic.
2. The vascular access procedure ends normally and the catheter is still in the tract. There is no bleeding.
3. The Z stitch is placed at the distal side of the insertion site and the proximal side of the access site.
4. Beneath the twist device 160 is a low power magnet 196 used to hold the powder in place within cavity 172. The magnet 196 is covered by a soft foam 194.
5. Magnetic BioSeal Advanced powder 36a is poured into the inverted device 160.
6. The magnet 196 holds the powder 36a in place as it is turned right side up and slid over the catheter and the suture ends. The device 160 is pressed down against the skin and the powder 36a is compressed over the wound site and in intimate contact with the wound.
7. The interfacial pH drops to 2 as the powder absorbs cation-rich blood liquid.
8. The two ends of the Z-stitch suture are pulled tight.
9. The suture ends are tied around the twist device 164 in the device 160 and knotted.
10. The twist device 164 is rotated one to two revolutions until skin inversion occurs, congruent with removing the catheter.
11. Hemostasis is immediate; time to ambulation is 60 minutes.

Device 160 is the preferred embodiment because it eliminates any time lag between pulling the catheter (or sheath) and creating skin inversion. This tensioner 160 also simplifies the procedure because there is only one knot, not two and eliminates any messiness potential with the powder 36a as the powder 36a is held in place magnetically.

The suture tensioner 160 includes a PCD 162 and a suture tensioner 164 which are preassembled together so, to the practioner, it is a one piece device, not two. The suture tensioner 164 pulls the sutures through a central slot 176/178 and holds tension on the sutures for the entire length of patient recovery. This can be accomplished by wrapping the suture around a cylindrical surface 170 of the PCD 162 after the suture ends have been locked into locking notches 192, followed by manual rotation of the suture tensioner 164 by finger blades 186.

The PCD 162 is formed having upwardly facing teeth 168 which lockingly mesh with the downwardly facing teeth 184 formed on the suture tensioner 164. An access notch 174 is formed radially inwardly from the perimeter of the body 166 of the PCD 162 for providing access around the in-place catheter. A clearance notch 188 in the perimeter of body 180. By making the powder 36a magnetic by the addition of magnetized powder such as Magnetite, and by providing a magnet 196 positioned at the top of cavity 172 covered by a compressible foam layer 194, all of the magnetic hemostatic powder 36a is held within the cavity 172 as the device 160 is positioned over the in-place sutures around the wound and wound tract as previously described.

The device 160 holds constant pressure over the wound in the vessel while in use with no outside assistance from a clinician. The pressure is created by gathering the tissue from the area around the insertion site, pulling it together while maintaining or creating a force over the skin to create an inversion in the tissue. This inversion in the tissue pushes down on the vessel stopping the bleeding. The sutures penetrate the skin on either side of the wound in the vessel, pulling the sides of the vessel upwards and inwards, creating a deep inversion that pushes down over the wound in the vessel as previously described.

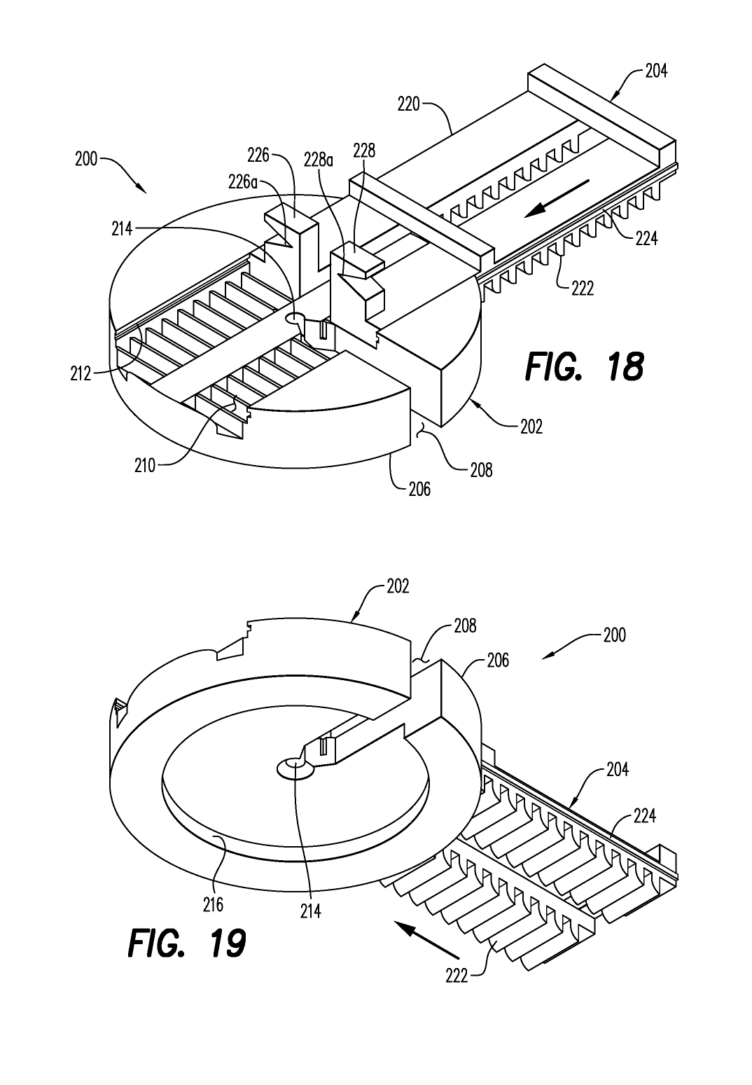

Referring now to FIGS. 18 and 19, a linear self-locking, one piece wound sealing device is there shown generally at numeral 200 and includes a PCD 202 and a suture tensioner 204 which are interengaged along mating linear grooves 212 and rails 224 so that the suture tensioner 204 is movable in the direction of the arrow with respect to the PCD 202.

The PCD 202 includes a cylindrical body 206 having a catheter clearance notch 208 formed radially inwardly from the perimeter to the center of the body 206 and also has a suture clearance hole 214 centrally therethrough. Upwardly facing teeth 210 of the PCD 202 interlock with the downwardly facing teeth 222 of the linear tract 220 of the suture tensioner 204. The suture ends upwardly extend from the central aperture 214 and lockingly engage within locking grooves 226a and 228a of locking members 226 and 228, respectively. Thereafter, linear movement of the suture tensioner 204 in the direction of the arrow applies tension in linear response to that movement to tension the Z-stitch positioned beneath the hemostatic powder cavity 216 in a fashion previously described.

### Clinical Trials

Clinical trials were conducted on randomly selected patients' legs, comparing the methodology of this disclosure to subcombinations thereof:
1. The first leg was subjected to a vascular procedure after which manual pressure only was applied.
   a. Time to hemostasis=20-30 minutes depending on blood chemistry
   b. Time to ambulate (TTA)=4-6 hours
   c. Complications=2%
2. The second leg was also subjected to the vascular procedure after which manual pressure and hemostatic powder were applied.
   a. Time to hemostasis=5-7 minutes
   b. TTA=2-4 hours
   c. Complications=2%
3. The third leg underwent the vascular procedure followed by application of the twisted suture procedure described hereinabove and hemostatic powder.
   a. Time to hemostasis=0 minutes
   b. TTA=1 hour
   c. Complications=0.1%
4. The trial is a random, prospective, multi-center trial using 7 doctors and four sites.
   a. Each doctor did 30 procedures of each leg.
5. Blood ACT<250

### Test Results

The results for the three conditions tested were:

Pressure only:
TTA=4.5 hr; Complications=2%; Patient rating=5

Pressure+BIOSEAL:
TTA=2.5 hr; Complications=2%; Patient rating=7

Suture Twist+BIOSEAL:
TTA=1.3 hr; Complications=0.5%; Patient rating=9

In a second experiment, the windlass was used without the haemostatic powder. Closure was achieved without manual pressure and the TTA was 1.5 hours; there was a 1% complication rate from infection; bandage changes were required to soak up oozing.

### Economic Data

The clinical trial also collected economic data and analyzed the economic impact of the three legs both as to cost and also as to revenue. The revenue analysis continued after the test period. The Twist+BIOSEAL significantly reduced the overall cost of the procedure.

The one-hour TTA was independent of the platelet count of the patient. Closure devices previously were used on about 30% of the cases; this skews by doctor. Some doctors used a closure device frequently (33% of all arterial accesses use closure devices), particularly when the patient had a low platelet count or other clotting compromise. Most doctors used closure devices infrequently. The most significant economic difference was the ability to schedule one additional case per day without overtime, a revenue increase of $2,000 per day per clinic. The increase in cost was $50/day for the suture twist kit 10.

While a number of exemplary aspects and embodiments have been discussed above, those of skill in the art will recognize certain modifications, permeations and additions and subcombinations thereof. It is therefore intended that the following appended claims and claims hereinafter introduced are interpreted to include all such modifications, permeations, additions and subcombinations that are within their true spirit and scope.

1-2. (canceled)

3. A wound sealing system for closing and arresting blood flow from and protecting a wound and wound tract after a vascular access procedure comprising:
a powder containment device (PCD) formed of flat plastic material sized to surround a skin area around the wound and a catheter of an I.V. within and extending from the wound tract, said PCD having a hole formed centrally therethrough providing access to the skin area and a Z-stitch around the wound;
a suture twisting member positioned against said PCD and configured to lockingly receive the ends of a suture exiting from two of the four suture holes whereupon rotation of said twisting member tightens the Z-stitch, closing the wound and wound tract, and arresting blood flow therefrom after the catheter is removed from the wound tract;
said hole being sized to receive a quantity of an anhydrous hemostatic agent sufficient to cover the wound and suture holes;
said PCD having a slot formed through a perimeter thereof for installation clearance around the catheter.

4. A wound sealing system for closing and arresting blood flow from and protecting a wound and wound tract after a vascular access procedure comprising:
a powder containment device (PCD) formed of plastic material and sized to cover a skin area around the wound and wound tract and a catheter of an I.V. within the wound tract, said PCD having a hole formed centrally therethrough sized to fit around four suture holes made in the skin area in forming a Z-stitch to close the wound;
a suture twisting member concentrically positioned against and atop said PCD and having spaced teeth which are engageable with mating teeth on said PCD which allow said twisting member to be rotated in only one direction relative to said PCD;
said twisting member having notches formed thereinto which are configured to lockingly receive the ends of a suture exiting from two of the four suture holes of the Z-stitch whereupon rotation of said twisting member tightens the Z-stitch, closing the wound and wound tract, and arresting blood flow therefrom after the catheter is removed from the wound tract;
said hole being sized to receive a quantity of an anhydrous hemostatic agent sufficient to cover the wound and suture holes;
said PCD having a slot formed through a perimeter thereof for installation clearance around the catheter.

5. A method of closing a wound tract of a vascular access site formed into a blood vessel comprising:
forming a skin inversion in alignment with and around the wound tract by suturing a Z-stitch into a skin area around the wound tract, the submerged portions of the Z-stitch extending into the skin area to a depth of the blood vessel and being oriented substantially perpendicular to the wound tract and centered over the vessel access site;
covering the wound and suture holes with a hemostatic powder;
tightening and knotting the ends of the suture together in an X configuration between two of the four suture holes formed by the suture;
twisting the suture ends together to torque and tension the Z-stitch such that the skin area is pulled together into the skin inversion to close the wound tract and arrest substantially all blood flowing therefrom;
removing a catheter from the blood vessel wound tract;
maintaining the twist on the suture ends for a time sufficient to completely arrest blood flow from the wound when suture tension is released.

6. The method of claim 5, further comprising:
applying pressure against the hemostatic powder over the wound as the catheter is removed from the blood vessel wound tract.

7. A method of closing a wound tract of a vascular access site when a catheter is removed from a blood vessel comprising:
creating semi-occlusive pressure along substantially an entire length of the wound tract by suturing a Z-stitch into a skin area around a wound and wound tract and into the skin area down to the blood vessel, the submerged portions of the Z-stitch being oriented substantially perpendicular to the wound tract and substantially centered over the vessel access site;
covering the wound and suture holes with a hemostatic powder;
tightening and knotting the exposed ends of the suture together in an X configuration between two of the four suture holes formed by the suture;
twisting the suture ends together to torque and tension the Z-stitch such that the skin area is pulled together into a skin inversion to close the wound tract and arrest substantially all blood flowing therefrom;
removing a catheter from the blood vessel wound tract;
maintaining the twist on the suture ends for a time sufficient to completely arrest blood flow from the wound when suture tension is released.

8. The method of claim 7, further comprising:
applying pressure against the hemostatic powder over the wound as the catheter is removed from the blood vessel wound tract.

9. A method of closing a wound tract formed by insertion of a catheter into a blood vessel at a vascular access site comprising:
closing the wound tract by suturing a Z-stitch into a skin area around the wound tract, the submerged portions of the Z-stitch extending into the skin area to a depth equal to a length of the wound tract and oriented substantially perpendicular to the wound tract and centered over the vessel access site;
covering the wound and suture holes with a hemostatic powder;
tightening and knotting the ends of the suture together in an X configuration between two of the four suture holes formed by the suture;
twisting the suture ends together to torque and tension the Z-stitch such that the skin area is pulled together into a skin inversion to close the wound tract and arrest substantially all blood flowing therefrom;
removing a catheter from the blood vessel wound tract;
maintaining the twist on the suture ends for a time sufficient to completely arrest blood flow from the wound when suture tension is released.

10. The method of claim 9, further comprising:
applying pressure against the hemostatic powder over the wound as the catheter is removed from the blood vessel wound tract.
